(19) United States
(12) Patent Application Publication
(10) Pub. No.: US 2025/0001162 A1
(43) Pub. Date: Jan. 2, 2025

(54) Apparatus Having Electrode Array Subassemblies Coupled Together By A Flexible Coupling

(71) Applicant: NOVOCURE GMBH, Root D4 (CH)

(72) Inventor: Elie Yaacobi, Haifa (IL)

(21) Appl. No.: 18/759,154

(22) Filed: Jun. 28, 2024

Related U.S. Application Data
(60) Provisional application No. 63/511,507, filed on Jun. 30, 2023.

(51) Int. Cl. A61N 1/04; A61N 1/36

(52) U.S. Cl. CPC A61N 1/0476; A61N 1/0484; A61N 1/36002; A61N 1/36034

(57) ABSTRACT

An electrode array assembly for providing tumor-treating fields includes first and second electrode array subassemblies, each of the first and second electrode array subassemblies including at least one electrode. The electrode array assembly further includes a flexible coupling extending between, and coupled to, the first and second electrode array subassemblies. The flexible coupling provides a spacing, measured along a surface of the electrode array assembly, between the first and second electrode array subassemblies.

### CROSS-REFERENCE TO RELATED APPLICATIONS

This application claims priority to and the benefit of the filing date of U.S. Provisional Patent Application No. 63/511,507, filed Jun. 30, 2023, the entirety of which is hereby incorporated by reference herein.

### FIELD

This application relates to apparatuses for providing Tumor Treating Fields.

### BACKGROUND

Tumor Treating Fields (TTFields) therapy is a proven approach for treating tumors using alternating electric fields at frequencies between 100-500 KHz. The alternating electric fields are induced by electrode assemblies (e.g., arrays of capacitively coupled electrodes, also called transducer arrays) placed on opposite sides of a target location in the subject's body. When an AC voltage is applied between opposing electrode assemblies, an AC current is coupled through the electrode assemblies and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

Further, treatment often requires use of multiple electrode arrays. Conventionally, each array requires its own cable, which can be undesirable for many reasons. For example, multiple cables can lead to complications from discomfort due to tangling of cables and/or, in some situations, pulling on or detachment of the arrays.

### SUMMARY

Disclosed herein, in one aspect, is an electrode array assembly including first and second electrode array subassemblies, each of the first and second electrode array subassemblies including at least one electrode. The electrode array assembly further includes a flexible coupling extending between, and coupled to, the first and second electrode array subassemblies. The flexible coupling provides a spacing, measured along a surface of the electrode array assembly, between the first and second electrode array subassemblies.

Also disclosed herein is a system comprising a plurality of electrode array assemblies including at least a first electrode assembly and a second electrode assembly, each of the plurality of electrode array assemblies comprising at least a first electrode array subassembly and a second electrode array subassembly and a flexible coupling extending between, and coupled to, the first and second electrode array subassemblies. The system further comprises at least one additional strap extending between the first electrode array subassembly of the first electrode assembly and the first electrode array subassembly of the second electrode assembly. Each of the first and second electrode array subassemblies comprises at least one electrode. The flexible coupling provides a spacing, measured along a surface of the electrode array assembly, between the first and second electrode array subassemblies.

In further aspects, methods of positioning the electrode array assemblies are disclosed. For example, a method includes positioning, on a body of a patient, an electrode array assembly including a plurality of electrode array subassemblies, the plurality of electrode array subassemblies including at least a first electrode array subassembly and a second electrode array subassembly. Each electrode array subassembly of the plurality of electrode array subassemblies includes at least one electrode. A flexible coupling extends between, and couples to, the first and second electrode array subassemblies. The flexible coupling provides a spacing, measured along a surface of the electrode array assembly, between the first and second electrode array subassemblies. In further aspects, the method can further include applying an electric field between the at least one electrode of the first electrode array subassembly and the at least one electrode of the second electrode array subassembly.

### BRIEF DESCRIPTION OF THE DRAWINGS

FIG. 1 is a schematic top view of an exemplary electrode array assembly for providing TTFields as disclosed herein.

FIG. 2 is a schematic top view of another exemplary electrode array assembly for providing TTFields as disclosed herein.

FIG. 3 is an exemplary cross-sectional view of the electrode array assembly of FIG. 1, taken at line A-A′, showing an exemplary electrode array subassembly.

FIG. 4 is another exemplary cross-sectional view of the assembly of FIG. 1, taken at line A-A′, showing an exemplary electrode array subassembly.

FIG. 5 is yet another exemplary cross-sectional view of the assembly of FIG. 1, taken at line A-A′, showing an exemplary electrode array subassembly.

FIG. 6 is an exemplary cross-sectional view of the assembly of FIG. 2, taken at line B-B′.

FIG. 7 is yet another exemplary cross-sectional view of the assembly of FIG. 1, taken at line A-A′, showing an exemplary electrode array subassembly.

FIG. 8 is yet another exemplary cross-sectional view of the assembly of FIG. 1, taken at line A-A′, showing an exemplary electrode array subassembly.

FIG. 9 is a block diagram of a system for using the electrode array assembly as disclosed herein.

FIG. 10 is a schematic front view of a system comprising an electrode array assembly with an additional strap in accordance with embodiments disclosed herein.

FIG. 11 is a schematic rear view of a system comprising an electrode array assembly with an additional strap in accordance with embodiments disclosed herein.

FIG. 12 is a schematic front view of a system comprising a pair of electrode array assembly with additional straps in accordance with embodiments disclosed herein.

FIG. 13 is a schematic rear view of a system comprising a pair of electrode array assembly with additional straps in accordance with embodiments disclosed herein.

FIG. 14 is a schematic front view of a system comprising a pair of electrode array assembly with additional straps in accordance with embodiments disclosed herein.

FIG. 15 is a schematic rear view of a system comprising a pair of electrode array assembly with additional straps in accordance with embodiments disclosed herein.

Various embodiments are described in detail below with reference to the accompanying drawings, wherein like reference numerals represent like elements.

### DETAILED DESCRIPTION

This application describes apparatuses (e.g., exemplary electrode array assemblies and/or treatment assemblies) that can be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

### Overview

Electrode arrays can be positioned on a patient with a target region therebetween for providing TTFields therapy. Conventionally, separate electrode arrays are individually positioned. However, positioning of the electrodes can be difficult, especially on the back of the torso. Improper positioning can lead to diminished effectiveness. Accordingly, difficulties in positioning may lead to loss of independence of the patient, requiring another individual such as a healthcare professional or care provider to position the electrode arrays. Further, each electrode array is conventionally associated with a respective cable. Accordingly, multiple arrays result in multiple cords that can be undesirable for aesthetic, comfort, and performance reasons.

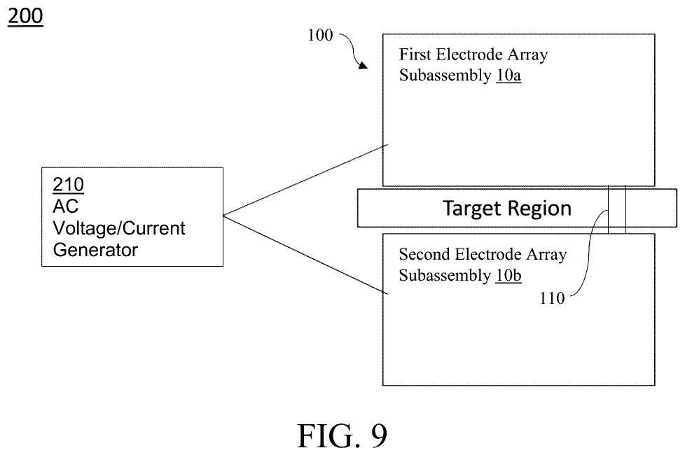

FIG. 9, further described herein, schematically depicts an exemplary system 200 for delivery of tumor treating fields using an electrode array assembly as disclosed herein. An electrode array assembly comprising two electrode subassemblies can be coupled together with a flexible coupling so that the distance therebetween is controllable and determined. In this way, the position of one electrode array subassembly can at least partly control the position of the other electrode array subassembly. Thus, for example, a first electrode array subassembly can be positioned on a more easily accessed location on the patient (e.g., the front of the torso of the patient), and a second electrode array subassembly can be positioned on a less accessible location on the patient (e.g., the back of the torso of the patient), using the length of the flexible coupling to properly position the second electrode array subassembly from the first electrode array subassembly.

### The Electrode Array Assembly

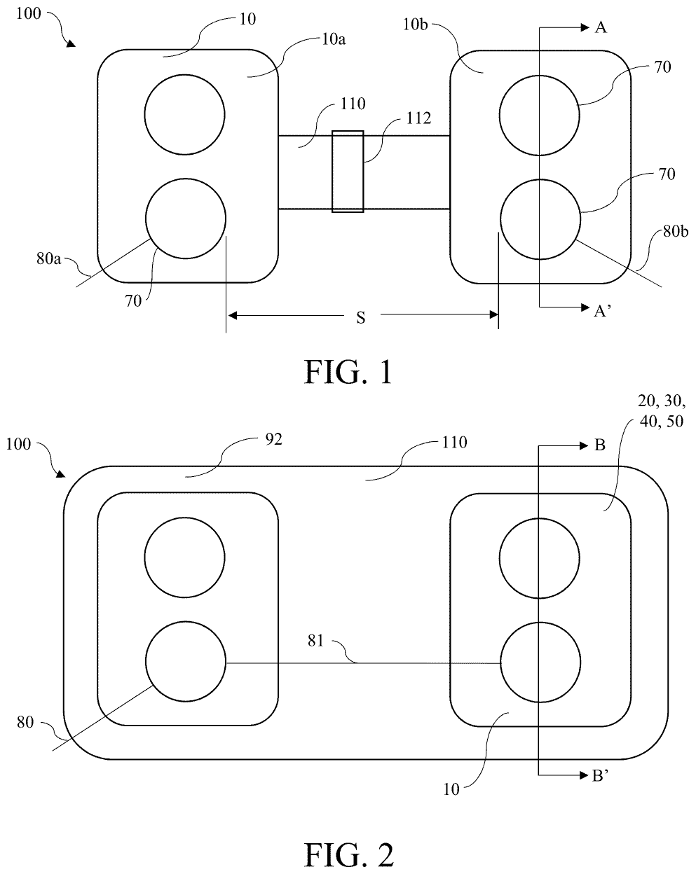

Disclosed herein, and with reference to FIGS. 1-2, is an electrode array assembly 100 comprising first and second electrode array subassemblies 10a,b, each of the first and second electrode array subassemblies 10a,b comprising at least one electrode 70. A flexible coupling 110 can extend between, and couple to, the first and second electrode array subassemblies 10a,b. The flexible coupling 110 can provide a spacing S, measured along a surface of the electrode array assembly, between the first and second electrode array subassemblies, as discussed further herein.

In exemplary aspects, each electrode 70 can comprise a metal pad. In some optional aspects, each electrode array subassembly 10 can comprise a plurality of electrodes 70. For example, in some aspects, the plurality of electrodes 70 can be arranged in a respective pattern. In other aspects, one or both of the first and second electrode array subassemblies 10a,b can have a single electrode 70.

In exemplary aspects, the flexible coupling 110 can provide a fixed, non-adjustable spacing, measured along the surface of the electrode array assembly, between the first and second electrode array subassemblies. Optionally, in these aspects, the fixed, non-adjustable spacing can be a patient-specific spacing. For example, the size of the patient can be taken into account for determining the patient-specific spacing.

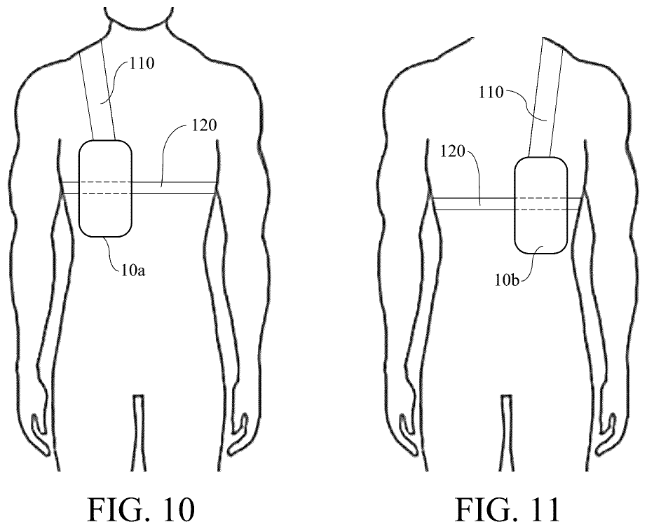

In some aspects, in which the fixed, non-adjustable spacing is a patient-specific spacing, the patient can be pre-fitted with an electrode array assembly and have the coupling specifically fitted and adjusted for the correct sizing and measurement to place the first and second electrode array subassemblies 10a,b in, respectively, first and second preferred positions in or on the patient. The first and second preferred positions may correspond to positions over two separate target locations (e.g., two separate tumors in the body). In further aspects, the fixed, non-adjustable spacing can be a patient-specific spacing that positions the first and second electrode array subassemblies over opposite sides of the same target location (e.g. a tumor) in or on the patient. For example, with further reference to FIGS. 10 and 11, the target location can be in the torso (e.g., in one lung). In some aspects, a single electrode array assembly 100 is positioned on the patient. In such aspects, the first electrode array subassembly 10a can be positioned on the chest of the patient, and the second electrode array subassembly 10b can be positioned on the back of the patient. FIG. 10 depicts the front of the patient (and patient's chest) and FIG. 11 depicts the rear of the patient (and patient's back). Accordingly, FIGS. 10 and 11 depict an electrode array assembly 100 positioned on the patient with two electrode array subassemblies (10a positioned on the front and 10b positioned on the rear) spaced apart by a flexible coupling 110. The flexible coupling 110 can provide a length so that if the first electrode array subassembly 10a is properly positioned on the chest of the patient, when the flexible coupling extends over the shoulder of the patient without slack, the second electrode array subassembly 10b is at the proper height on the back of the patient.

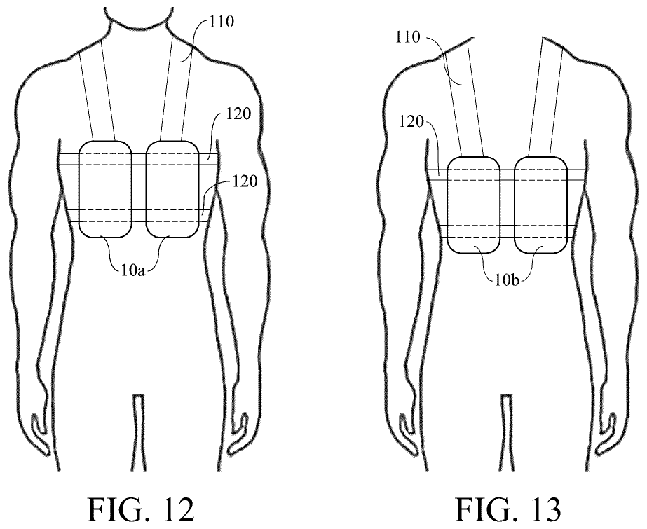

With reference to FIGS. 12 and 13, the target location can be in the torso (e.g., both lungs). In some aspects, a plurality of electrode array assemblies 100 (for example, two electrode array assemblies 100, as shown in FIGS. 12, 13) are positioned on the patient. FIG. 12 depicts the front of the patient (and patient's chest) and FIG. 13 depicts the rear of the patient (and patient's back). In FIGS. 12 and 13, two electrode array assemblies 100 are positioned on the patient, each one having two electrode array subassemblies (10a positioned on the front and 10b positioned on the rear), in each case the two electrode array subassemblies 10a, 10b are spaced apart by a flexible coupling 110. As described above, the flexible coupling 110 can provide a length so that, for each electrode array assembly 100, if the first electrode array subassembly 10a is properly positioned on the chest of the patient, when the flexible coupling extends over the shoulder of the patient without slack, the second electrode array subassembly 10b is at the proper height on the back of the patient. Accordingly, in this example, each electrode array assembly 100 can position the first and second electrode array subassemblies over opposite sides of a target location (e.g. a tumor), in this case each electrode array assembly 100 targeting a different location (e.g. two tumors, one in each lung). In some optional aspects, the flexible coupling 110 can permit adjustment of the spacing, measured along the surface of the electrode array assembly 100, between the first and second electrode array subassemblies 110a,b. In some exemplary embodiments, the flexible coupling 110 can comprise an adjustable strap. For example, the flexible coupling 110 can comprise at least one of: an adjustable slide strap adjuster 112; or hook material on a first portion of the flexible coupling and loop material on a second portion of the flexible coupling. An example of a hook and/or loop fastener is a Velcro® hook and loop type fastener (available from Velcro Companies of Manchester, NH). In some aspects, the flexible coupling 110 can have a length that permits simultaneous positioning of the first electrode array subassembly on a front portion of a torso of a patient and the second electrode array subassembly on a back portion of the torso of the patient. Accordingly, for a given patient, an adjustable flexible coupling 110 can be adjusted to an appropriate length initially and then utilized for repeated use by that patient without further adjustment.

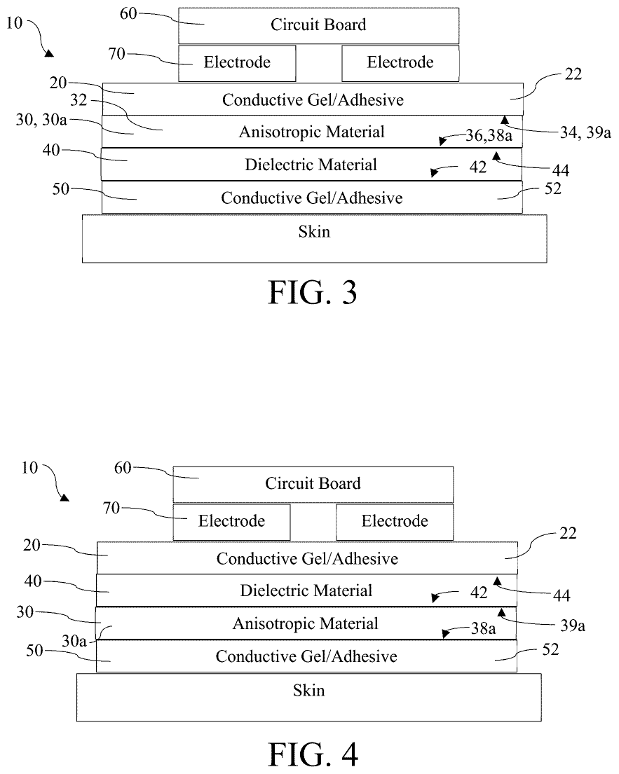

In some aspects, the spacing S, measured along the surface of the electrode array assembly 100, between the first and second electrode array subassemblies 10a,b is a shortest distance, measured along the surface of the electrode array assembly, between any electrode of the at least one electrode of the first electrode array subassembly and any electrode of the at least one electrode of the second electrode array subassembly (for example, between the closest electrodes 70 of the respective electrode array subassemblies 10a,b, as shown in FIG. 1). In some aspects, the spacing S, measured along the surface of the electrode array assembly 100, between the first and second electrode array subassemblies 10a,b is a shortest distance, measured along the surface of the electrode array assembly, between the perimeter of the first electrode array subassembly and the perimeter of the second electrode array subassembly. In some aspects, for which the electrode array assembly 100 comprises a layer of anisotropic material 30 (FIGS. 3-6, 8), the spacing S, measured along the surface of the electrode array assembly 100, between the first and second electrode array subassemblies 10a,b is a shortest distance, measured along the surface of the electrode array assembly, between the perimeter of the areal footprint of the layer of anisotropic material of the first electrode array subassembly and the perimeter of the areal footprint of the layer of anisotropic material of the second electrode array subassembly. In various aspects, the spacing, measured along the surface of the electrode array assembly, between the first and second electrode array subassemblies 10a,b can be from about 0.05 m to about 1.5 m. In exemplary aspects, said spacing can be at least 0.3 meters. In further exemplary aspects, said spacing can be at least 0.5 meters, or at least 0.75 meters, or at least 1 meter, or from about 0.5 meters to 1.5 meters.

Referring to FIG. 1, in some aspects, the electrode array assembly 100 can comprise a first electrical lead 80a and a second electrical lead 80b, wherein each electrode 70 of the first electrode array subassembly 10a can be in electrical communication with the first electrical lead 80a, and each electrode of the second electrode array subassembly 10b can be in communication with the second electrical lead 80b.

Referring to FIG. 2, in some optional aspects, the electrode array assembly 100 can include only a single cable 80. For example, electrical leads that provide current to both the first and second electrode array subassemblies 10a,b can extend through the cable 80. Accordingly, in some aspects, at least one electrical lead 81 can extend between the first and second electrode array subassemblies 10a,b so that the single cable 80 can provide current to both the first and second electrode array subassemblies 10a,b. In other aspects (not shown), the single cable 80 can branch to extend to both of the first and second electrode array subassemblies 10a,b. In this way, fewer cables can be needed for providing treatment. This can contrast to conventional systems, in which each electrode array requires its own cable, which can lead to complications from discomfort due to tangling of cables and/or pulling on or detachment of the arrays.

Referring also to FIG. 3, each electrode array subassembly 10 can comprise a flexible circuit 60 (e.g. flex circuit or printed circuit board) that extends between each electrode 70 of the electrode array subassembly. In further aspects, the electrode array subassembly 10 can comprise a printed circuit board (PCB).

Referring to FIGS. 2 and 6, in some aspects, the flexible coupling 110 can comprise a cover material 92 that extends over an outwardly facing side 94 of each of the electrode array subassemblies 10 (e.g. first and second electrode array subassemblies 10a,b).

In other aspects, and with reference to FIG. 1, the flexible coupling 110 can comprise a strap. The strap can have a thickness and a width that is greater than the thickness. For example, the width of the strap can be at least 5 times greater than the thickness of the strap. The width of the strap can be at least 1 centimeter, or at least 2 centimeters, or at least 5 centimeters. It is contemplated that the strap having such a width can inhibit undesired twisting of the flexible coupling that could affect the spacing between the first and second electrode array subassemblies 10a,b. Further, the strap having a sufficient width can inhibit angular offsetting of the first and second electrode array subassemblies 10a,b (e.g., about an axis that extends out of the page in FIG. 1). For example, rather than having an attachment point to the first and second electrode array subassemblies 10a,b that could permit angular pivoting, the strap can have an elongate region of attachment to the strap (e.g., a line) that extends perpendicular to a length of the strap, thereby minimizing angular pivoting of the first and second electrode array subassemblies.

Referring to FIGS. 10 and 11 (also FIGS. 12-15), in some optional aspects, at least one additional strap 120 can assist in positioning and securing the electrode array subassemblies 10a,b. The additional strap 120 can extend between the first and second electrode array subassemblies 10a,b of a single electrode array assembly 100 on a patient (FIGS. 10, 11). For example, in some embodiments, the at least one additional strap 120 can be configured to extend around the torso of the patient (for example, horizontally in FIGS. 10, 11). The at least one additional strap 120 can provide additional confirmation of proper positioning of the first and second electrode array subassemblies 10a,b. Further, the at least one additional strap 120 can secure the first and second electrode array subassemblies 10a,b in place on the patient.

In some aspects, the at least one additional strap can comprise a single strap. In some embodiments, a single (horizontal) additional strap 120 extending around the torso may help position and secure a single electrode array assembly 100 on a patient. The single additional strap can couple to an upper portion, or lower portion, or (as shown, FIGS. 10,11) a central portion of the first and second electrode array subassemblies 10a,b. Alternatively, in some embodiments, a plurality of additional straps 120 can extend around the torso and may help position and secure a single electrode array assembly 100 on a patient in similar fashion.

In some embodiments, a single (horizontal) additional strap 120 extending around the torso may help position and secure two electrode array assemblies 100 on a patient. For example, the single additional strap 120 may couple to the lower portion of the first and second electrode array subassemblies 10a,b for two electrode array assemblies 100. In other aspects, a plurality of (e.g., a pair of) additional straps 120 can couple to two electrode array assemblies 100 (FIGS. 12, 13). For example, a first strap can couple to an upper portion of the first and second electrode array subassemblies 10a,b of the two electrode array assemblies 100, and a second strap can couple to a lower portion of the first and second electrode array subassemblies 10a,b of the two electrode array assemblies 100.

In some aspects, the at least one additional strap 120 can be integral to the electrode array assembly 100 (e.g., via fusion, integral formation, or stitching). In other aspects, the at least one additional strap 120 can be a separate element that is removably coupled to the electrode array assembly 100. For example, the at least one additional strap 120 can comprise hook or loop fastener that is configured to couple to a corresponding hook or loop fastener of the electrode array assembly 100 or, for example, a nonwoven backing of the electrode array assembly. In other aspects, the at least one additional strap 120 can couple to the electrode array assembly 100 via adhesive.

In still additional aspects, the at least one additional strap 120 can be configured to extend across the electrode array assembly 100 (e.g., horizontally) and compressively retain the electrode array assembly against the patient. In further aspects, the at least one strap can be elastic and can couple to itself (e.g., via adhesive or a fastener), thereby forming a loop that can wrap around a patient to compress the electrode array assembly against the patient. For example, the at least one additional strap 120 can comprise a self-adhering bandage, such as Coban® bandage provided by 3M of Minneapolis, MN. In yet further exemplary aspects, the electrode array assembly 100 can define at least one opening (e.g., a slot) that receives the at least one additional strap 120 therethrough. Again, the compressive strap can position and secure a single electrode array assembly on a patient or two electrode array assemblies on a patient. One or more (for example, two) compressive straps may be used in similar fashion to that described above.

As discussed above, in some embodiments, a treatment system can comprise a plurality of (e.g., a pair of) electrode array assemblies 100. Optionally, the pair of electrode array assemblies 100 can be separate elements. In other aspects, the pair of electrode array assemblies 100 can be coupled together. Optionally, a single electrical lead can provide power to both electrode array assemblies 100. For example, the electrode array assemblies may be linked via a hub so that an AC voltage or AC current generator can generate electric fields for a plurality of electrode array assemblies. For example, the hub can comprise an input port that electrically couples to an AC voltage or AC current generator. The hub can further comprise a plurality of outlet ports that electrically couple to respective electrode array assemblies (or electrode array subassemblies) to provide electrical communication between the AC voltage or AC current generator and the coupled electrode array assemblies (or electrode array subassemblies). The hub can permit the AC voltage or AC current generator to generate electrical fields at each of the electrode array assemblies. In exemplary aspects, the hub can further permit the AC voltage or AC current generator to selectively generate electrical fields between one or more electrodes of at least two electrode array subassemblies of any of the electrode array assemblies coupled to the hub. In further aspects, the hub can permit the AC voltage or AC current generator to selectively generate electrical fields between at least two electrodes of a single electrode array subassembly. Optionally, in these aspects, electrical fields can be applied between the first and second electrode array subassemblies 10a,b of a single electrode array assembly 100. In other aspects, electrical fields can be applied between the first electrode array subassemblies 10a of a first electrode array assembly and the second electrode array subassembly 10b of a second electrode array subassembly (e.g., between the left chest and right back, or between the right chest and left back).

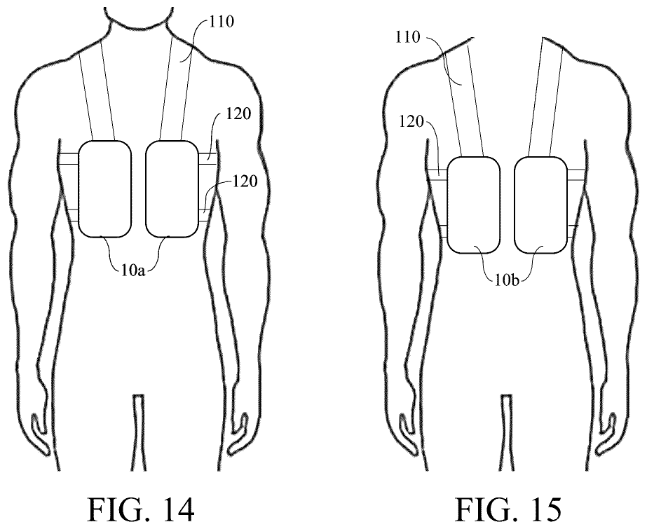

In some aspects, each strap of the at least one additional strap 120 can extend to (optionally, couple to) each of the plurality of electrode array assemblies 100. In other aspects, and with reference to FIGS. 14 and 15, the at least one additional strap 120 can extend only between the first and second electrode array subassemblies 10a,b of a single electrode array assembly 100. In this embodiment, each additional strap 120 does not extend all the way around the torso, but simply links from chest to back around one side. In these embodiments, the additional strap 120 may be supplied with an adhesive to adhere the additional strap 120 to the body.

Disclosed herein, and with reference to FIGS. 2-6, are exemplary electrode array subassemblies 10 that illustrate further aspects of the disclosed electrode array assembly 100. The electrode array subassembly 10 can comprise an outer adhesive layer 20. The outer adhesive layer 20 can comprise a conductive gel or conductive adhesive 22. The electrode array subassembly 10 can further comprise at least one layer of anisotropic material 30, a layer of dielectric material 40, and a skin contact layer 50. The inclusion of a layer of dielectric material can form a capacitive structure. The skin contact layer 50 can comprise a conductive gel or conductive adhesive 52. The layer of anisotropic material 30 can be a layer of nonmetallic anisotropic material.

In some embodiments, the layer of dielectric material 40 can be positioned between the electrodes and the outer adhesive layer 20. In some embodiments, the at least one layer of anisotropic material 30 and the layer of dielectric material 40 can be positioned between the outer adhesive layer 20 and the skin contact layer 50.

The circuit board 60 can be electrically coupled to the outer adhesive layer 20. In some optional aspects, the circuit board 60 can be electrically coupled to the outer adhesive layer 20 through one or more conductive elements (e.g., electrodes 70) that are in electrical contact with the circuit board 60. In FIGS. 3-6, two electrodes 70 are shown, but additional electrode elements can be included in each electrode array subassembly 10.

In some aspects, the layer of anisotropic material 30 can comprise a sheet of anisotropic material 32 having a rear face 34 and a front face 36 (the front face facing toward the subject's skin). The sheet of anisotropic material 32 can have a first thermal conductivity in a direction that is perpendicular to the front face 36. In some aspects, the thermal conductivity of the sheet in directions that are parallel to the front face can be more than two times higher than the first thermal conductivity. In further aspects, the sheet of anisotropic material 32 can have a first resistance in a direction that is perpendicular to the front face, and the resistance of the sheet in directions that are parallel to the front face can be less than half of the first resistance.

In some optional aspects, the first layer of anisotropic material 30a can comprise graphite.

Optionally, the first layer of anisotropic material 30a can comprise a synthetic graphite.

In further optional aspects, the first layer of anisotropic material 30a can comprise a sheet of pyrolytic graphite or graphitized polymer film.

In further optional aspects, the first layer of anisotropic material 30a can comprise graphite foil. For example, optionally, the first layer of anisotropic material can comprise graphite foil made from compressed high purity exfoliated mineral graphite.

The layer of dielectric material 40 can have a skin-facing surface 42 and an opposing outwardly facing surface 44. The first layer of anisotropic material 30a can have a skin-facing surface 38a and an opposing outwardly facing surface 39a. In some aspects, and as shown in FIG. 3, the outwardly facing surface 44 of the dielectric material 40 can contact the skin-facing surface 38a of the first layer of anisotropic material 30a. In some embodiments, and as shown in FIG. 3, the outwardly facing surface 39a of the first layer of anisotropic material 30a can contact the outer adhesive layer 20. In further aspects, and as shown in FIG. 3, the skin-facing surface 42 of the layer of dielectric material 40 can contact the skin contact layer 50.

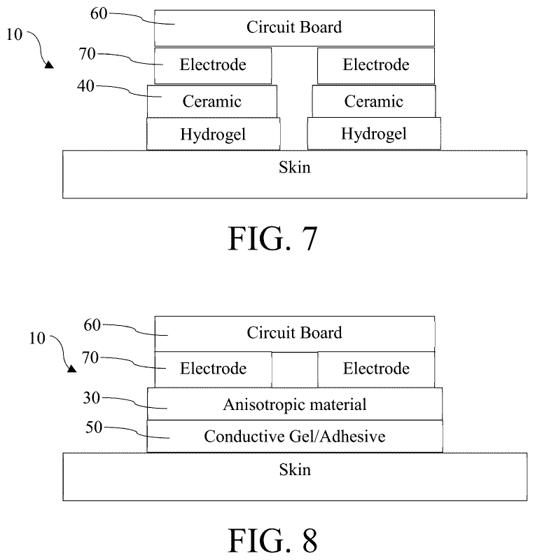

In some aspects, the outwardly facing surface 44 of the dielectric material 40 can directly contact the at least one electrode 70 (e.g., FIG. 7). In other aspects, an outer adhesive layer 20 can be present between the dielectric material 40 and the at least one electrode 70. In these aspects, the outwardly facing surface 44 of the dielectric material 40 can contact the outer adhesive layer 20 (FIG. 4). In some aspects, and as shown in FIG. 4, the skin-facing surface 42 of the dielectric material 40 can contact the outwardly facing surface 39a of the first layer of anisotropic material 30a. In some embodiments, the skin-facing surface 38a of the first layer of anisotropic material 30a can contact the skin contact layer 50. The embodiments of FIG. 4 show the relative positioning of the anisotropic material layer 30a and the dielectric material layer 40 reversed between the outer adhesive layer 20 and the skin contact layer 50 compared with the FIG. 3 embodiments. In some such FIG. 4 embodiments, the relative positioning of the other components in the electrode array subassembly 10, such as the circuit board 60 and the electrodes 70, can be unchanged.

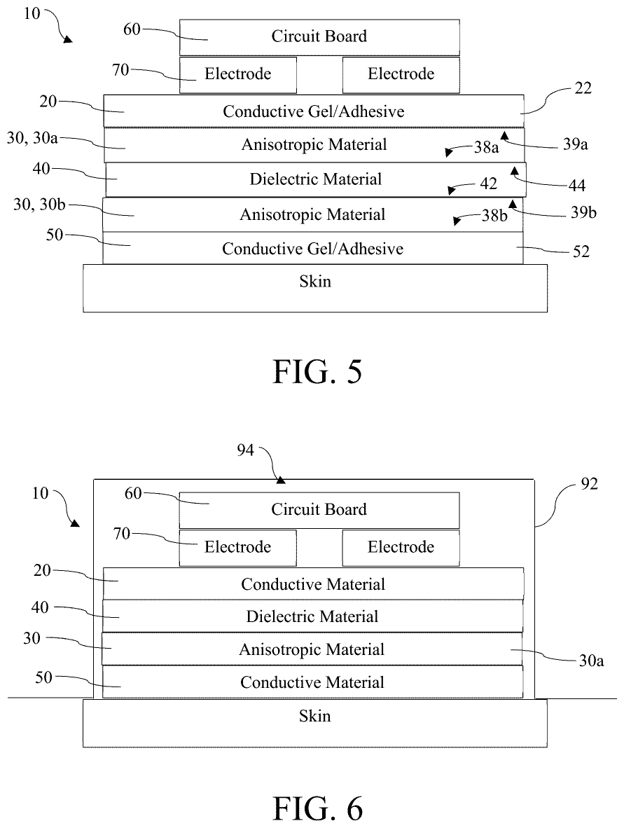

Referring to FIG. 5, in some optional aspects, the at least one layer of anisotropic material 30 can comprise a second layer of anisotropic material 30b. The layer of dielectric material 40 can be positioned between the first and second layers of anisotropic material 30a,b. The second layer of anisotropic material 30b can have a skin-facing surface 38b and an opposing outwardly facing surface 39b. In some aspects, the outwardly facing surface 39a of the first layer of anisotropic material 30a can contact the outer adhesive layer 20. In further aspects, the skin-facing surface 42 of the layer of dielectric material 40 can contact the outwardly facing surface 39b of the second layer of anisotropic material 30b. In still further aspects, the skin-facing surface 38b of the second layer of anisotropic material 38b can contact the skin contact layer 50.

In some optional aspects, the layer of dielectric material 40 can be positioned between and contact both the first and second layers of anisotropic material 30a,b (FIG. 5). In these aspects, other than the 3-layer sandwich structure (first layer of anisotropic material 30a—dielectric material layer 40—second layer of anisotropic material 30b), the relative positioning of the other components in the electrode array subassembly 10, such as the circuit board 60 and the electrodes 70, can be unchanged.

In some optional aspects, the electrode array subassembly 10 can comprise a wire 80 (FIG. 2) that is electrically coupled to the outer adhesive layer 20. Optionally, in these aspects, the electrode array subassembly 10 does not comprise a circuit board 60 or flex circuit. Optionally, the wire 80 can be coupled to the outer adhesive layer 20 via one or more electrodes 70 or via a metal layer. Optionally, and in the alternative, the electrode array subassembly 10 does not comprise a metal pad or a metal layer. Optionally, the wire 80 can be coupled to the outer adhesive layer 20 via a circuit board 60 or flex circuit. Such embodiments can exist for each of the embodiments described herein.

Referring to FIG. 6, the electrode array subassembly 10 can comprise at least one layer of anisotropic material 30 and a layer of dielectric material 40. The layer of dielectric material 40 can contact at least a first layer 30a of the at least one layer of anisotropic material 30.

The at least one layer of anisotropic material 30 and the layer of dielectric material 40 can be positioned between opposed layers of conductive materials (e.g., between the outer adhesive layer 20 and the skin contact layer 50). In various aspects, the conductive materials can optionally comprise a conductive gel or conductive adhesive. In further aspects, the conductive materials can comprise conductive grease. In some aspects, one of the outer adhesive layer 20 and the skin contact layer 50 can comprise conductive grease and the other of the outer adhesive layer 20 and the skin contact layer 50 can comprise a conductive gel or conductive adhesive. In these aspects, a cover 92 (e.g., a bandage, plaster, or other covering structure) can retain the subassembly 10 in a stacked arrangement and against the skin of the patient. Optionally, a bandage or other cover 92 may be utilized in any of the embodiments described herein. Other than replacing the conductive gel or conductive adhesive of the outer adhesive layer with a conductive grease, and the optional addition of a bandage or cover 92, the FIG. 6 embodiment resembles that of the FIG. 4 embodiment, and the relative positioning of the other components in the electrode array subassembly 10, such as the circuit board 60 and the electrodes 70 (e.g., metal pads), can be unchanged. Indeed, the replacement of the conductive gel or conductive adhesive of the outer adhesive layer with a conductive grease, and/or the optional addition of a bandage or cover 92, can also be employed as an additional embodiment for any and all of the other embodiments described herein. Moreover, the replacement of the conductive gel or conductive adhesive of the skin contact layer with a conductive grease, and/or the optional addition of a bandage or cover 92, can also be employed as an additional embodiment for any and all of the other embodiments described herein.

Exemplary embodiments disclosed herein incorporate into the electrode array subassembly 10 a sheet of material having anisotropic thermal properties and/or anisotropic electrical properties (referred to herein also as the layer of anisotropic material 30). If the sheet of material has anisotropic thermal properties (e.g., greater in-plane thermal conductivity than perpendicular to the plane), then the sheet spreads the heat out more evenly over a larger surface area. If the sheet of material has anisotropic electrical properties (e.g., greater in-plane electrical conductivity than perpendicular to the plane; or, conversely, lower in-plane resistance than perpendicular to the plane), then the sheet spreads the current out more evenly over a larger surface area. In each case, this lowers the temperature of the hot spots and raises the temperature of the cooler regions when a given AC voltage is applied to the electrode array subassembly. Accordingly, the current can be increased (thereby increasing the therapeutic effect) without exceeding the safety temperature threshold at any point on the subject's skin.

In some embodiments, the anisotropic material is anisotropic with respect to electrical conductivity properties. In some embodiments, the anisotropic material is anisotropic with respect to thermal conductivity properties. In some embodiments, the anisotropic material is anisotropic with respect to both electrical conductivity properties and thermal conductivity properties.

The anisotropic thermal properties include directional thermal properties. Specifically, the sheet has a first thermal conductivity in a direction that is perpendicular to its front face. And the thermal conductivity of the sheet in directions parallel to the front face is more than two times higher than the first thermal conductivity. In some preferred embodiments, the thermal conductivity in the parallel directions is more than ten times higher than the first thermal conductivity. For example, the thermal conductivity of the sheet in directions that are parallel to the front face can be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the sheet has a first resistance in a direction that is perpendicular to its front face. And resistance of the sheet in directions parallel to the front face is less than the first resistance. In some preferred embodiments, the resistance in the parallel directions is less than half of the first resistance or less than 10% of the first resistance. For example, the resistance of the sheet 70 in directions that are parallel to the front face can be less than: 75%, 50%, 40%, 30%, 20%, 10%, 5%, 1%, 0.5%, or even less than 0.1% of the first resistance.

In some embodiments (e.g., when the sheet of anisotropic material is a sheet of pyrolytic graphite), the sheet of anisotropic material has both anisotropic electrical properties and anisotropic thermal properties.

Use of nonmetallic anisotropic material is particularly advantageous in situations where preventing the transfer of ions into a subject's body is desirable. More specifically, using a metallic sheet could result in the transfer of ions into a subject's body. For all of the embodiments herein, alternative embodiments exist that do not include the sheet of anisotropic material.

In some aspects, the layer of dielectric material 40 can have a dielectric constant of at least 10. In some optional aspects, the layer of dielectric material 40 can comprise a high dielectric constant polymer (dielectric constant of at least 10). In alternative aspects, the dielectric material 40 can be a ceramic material. In alternative aspects, the dielectric material 40 can be a metal oxide, e.g. Al₂O₃, which can optionally be applied by chemical vapor deposition, CVD, onto a substrate and is sufficiently flexible as a thin film.

In various optional aspects, the layer of dielectric material 40 can have a dielectric constant ranging from 10 to 50,000.

In some preferred embodiments, the high dielectric polymer material 40 comprises poly (vinylidene fluoride-trifluoroethylene-chlorotrifluoroethylene) and/or poly (vinylidene fluoride-trifluoroethylene-1-chlorofluoroethylene). Those two polymers are abbreviated herein as “Poly (VDF-TrFE-CTFE)” and “Poly (VDF-TrFE-CFE),” respectively. These embodiments are particularly advantageous because the dielectric constant of these materials is on the order of 40. In some embodiments, the polymer layer can be poly (vinylidene fluoride-trifluoroethylene-chlorotrifluoroethylene-chlorofluoroethylene) or “Poly (VDF-TrFE-CTFE-CFE).”

In some embodiments, the terpolymer used in the insulating polymer layer can comprise VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer

FIG. 7 illustrates another embodiment of an electrode array subassembly 10 in accordance with embodiments disclosed herein. In some aspects, one or both of the electrode array subassemblies 10 can comprise a respective ceramic material positioned over each electrode 70 that serves as a dielectric material 40. In further optional aspects, a skin contact layer 50 can comprise hydrogel. Optionally, a respective skin contact layer (e.g., hydrogel or conductive gel/adhesive) can be associated with each electrode 70. For example, in exemplary aspects, the sheet of anisotropic material 30 can be omitted.

Further, aspects of the various embodiments can be combined to provide further aspects of the present disclosure. For example, aspects and elements of the embodiments described with reference to FIGS. 3-6 can be added or interchanged with the elements described in FIG. 7. For example, in another aspect, the electrode array subassembly 10 can comprise a ceramic material as the dielectric material 40 and a conductive adhesive/gel 50. Optionally, in these aspects, a respective ceramic material can be positioned over each electrode 70. In still other aspects, and, as illustrated in FIG. 8, the electrode array subassembly 10 can omit (be free of) a dielectric material. Optionally, other components as outlined herein may be present in the embodiments of FIG. 8.

### Method of Using the Electrode Array Assembly

A method can comprise positioning the electrode array assembly 100 of any one of the preceding claims on a body of a patient. The first electrode array subassembly can be applied on a first location of the body of the patient, and the second electrode array subassembly can be applied on a second location of the body of the patient that is spaced along the surface of the body by the spacing S, measured along the surface of the electrode array assembly, between the first and second electrode subassemblies. In some aspects, upon applying the second electrode subassembly, the flexible coupling contacts the body of the patient along substantially an entire length of the flexible coupling. In various aspects, the electrode array assembly 100 can be positioned so that a target region (e.g., a tumor) is between the first and second electrode array subassemblies 10a,b. For example, the first location can be on a front of a torso of the body of the patient, and the second location can be on a back of the torso of the body of the patient. In another example, the first location can be on the subject's skin at the right of the subject's head, and the second location can be on the subject's skin at the left of the patient's head.

In aspects in which the flexible coupling comprises an adjustable strap, the length of the adjustable strap can be adjusted. In some aspects, adjustment of the strap can be performed by a medical professional for a fitting. In other aspects, the patient or a caretaker can adjust the strap.

FIG. 9 illustrates an exemplary system 200 for applying electrical fields using the electrode array assembly 100. The electrode array assembly 100 can be used to apply an electric field between the at least one electrode of the first electrode array subassembly 10a and the at least one electrode of the second electrode array subassembly 10b. An AC voltage or AC current generator 210 can be in communication with each electrode array subassembly 10. The AC voltage or AC current generator 210 can be configured to generate alternating electric fields through the target region (e.g., a tumor).

In some optional aspects, applying the electric field can comprise applying an alternating electric field having an alternating current waveform at frequencies in a range from about 50 kHz to about 1 MHz. Optionally, in these aspects, applying the alternating electric field can generate an electric field having an electric field intensity in the range of about 0.1 V/cm to about 10 V/cm.

In some embodiments, the frequency of the alternating voltage is between 50 kHz and 1 MHz, or between 100 kHz and 500 kHz. In some aspects, the AC voltage generator can be controlled by a controller. The controller can use temperature measurements to control the amplitude of the current to be delivered via the electrode array subassemblies 10 in order to maintain temperatures below a safety threshold (e.g., 41° C.). This can be accomplished, for example, by measuring a first temperature of the first electrode element, measuring a second temperature of the second electrode element, and controlling the applying of the alternating voltage based on the first temperature and the second temperature, as described below.

More specifically, temperature sensors (e.g., thermistors) can be positioned in thermal contact with respective electrode elements within each of the electrode array subassemblies 10. The temperature sensors can measure respective first and second temperatures (e.g., at first and second electrode elements in the first and second electrode array subassemblies 10a, 10b, respectively), and the controller can control the output of the AC voltage generator based on these temperatures. The use of further temperature sensors positioned at additional electrode elements can measure temperatures at a plurality of electrode elements in the transducer array, and the controller can control the current applied to each electrode element according to a delta temperature compared to the threshold temperature (e.g., 41° C.), and thereby balance any temperature hot-spots on the array.

As discussed above, a flexible coupling 110 can extend between, and couple to, the first and second electrode array subassemblies 10a,b (FIG. 9). The flexible coupling 110 can provide a spacing S, measured along a surface of the electrode array assembly, between the first and second electrode array subassemblies.

Electrode Array Subassemblies Having Layers that Comprise a Conductive Adhesive Composite

Optionally, the outer adhesive layer 20 and/or the skin contact layer 50 can comprise hydrogel. It is further contemplated that the outer adhesive layer 20 and/or the skin contact layer 50 can comprise conductive adhesive composites (described further below) rather than hydrogel.

In exemplary aspects, the conductive adhesive composite can comprise a dielectric material and conductive particles dispersed within the dielectric material. In some embodiments, at least a portion of the conductive particles define a conductive pathway through a thickness of the conductive adhesive composite. In some aspects, the dielectric material is a polymeric adhesive. Optionally, in these aspects, the polymeric adhesive can be an acrylic adhesive. In some aspects, the conductive particles can comprise carbon. Optionally, in these aspects, the conductive particles can comprise graphite powder. Additionally, or alternatively, the conductive particles can comprise carbon flakes. Additionally, or alternatively, the conductive particles can comprise carbon granules. Additionally, or alternatively, the conductive particles can comprise carbon nanotubes. Additionally, or alternatively, the conductive particles can comprise carbon nanowires. Additionally, or alternatively, the conductive particles can comprise carbon fibers. Additionally, or alternatively, the conductive particles can comprise carbon black powder. In further aspects, the conductive adhesive composite further comprises a polar material (e.g., a polar salt). The polar salt can be a quaternary ammonium salt, such as a tetra alkyl ammonium salt. Exemplary conductive adhesive composites, as well as methods for making such conductive adhesive composites, are disclosed in U.S. Pat. Nos. 8,673,184 and 9,947,432, which are incorporated herein by reference for all purposes. In exemplary aspects, the conductive adhesive composite can be a dry carbon/salt adhesive, such as the OMNI-WAVE adhesive compositions manufactured and sold by FLEXCON (Spencer, MA, USA). In other exemplary aspects, the conductive adhesive composite can be an electrically conductive adhesive, such as the ARcare® 8006 electrically conductive adhesive composition manufactured and sold by Adhesives Research, Inc. (Glen Rock, PA, USA).

In exemplary aspects, the outer adhesive layer 20 and/or the skin contact layer 50 does not comprise hydrogel.

In still further aspects, the conductive adhesive composite layer has a thickness ranging from about 30 μm to about 2000 μm, such as from 30 μm to about 200 μm. Optionally, the conductive adhesive composite in the outer adhesive layer can have a thickness ranging from about 30 μm to about 2000 μm, or from about 50 μm to about 1000 μm, or from about 50 μm to about 200 μm, or from about 70 μm to about 150 μm. Optionally, the conductive adhesive composite in the skin contact layer can have a thickness ranging from about 30 μm to about 100 μm, or from about 30 μm to about 70 μm, or from about 40 μm to about 60 μm, or from about 45 μm to about 55 μm.

In still further aspects, the conductive adhesive composite does not comprise water.

Optionally, in exemplary aspects, the electrode array subassembly can further comprise a release liner that covers the skin contact layer. In these aspects, it is contemplated that, prior to use, the electrode array subassembly can be provided with the release liner to ensure that the skin contact layer does not adhere to undesirable surfaces or locations. Immediately prior to use, the release liner can be removed, and the skin contact layer can be positioned in contact with the skin of the patient.

It is further contemplated that embodiments that include the sheet of anisotropic material can additionally aid in avoiding or reducing overheating of the electrodes and associated discomfort on the skin by dissipating both electrical current and heat in a lateral (in-plane) direction rather than passing directly through the layer (in a direction perpendicular to the plane of the skin contact layer) in a concentrated manner. In some embodiments, the layer of anisotropic material may be present as, or may compromise, a laminate having a layer of conductive adhesive, a layer of anisotropic material, and a layer of conductive adhesive.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: An electrode array assembly comprising:
first and second electrode array subassemblies, each of the first and second electrode array subassemblies comprising at least one electrode; and
a flexible coupling extending between, and coupled to, the first and second electrode array subassemblies, wherein the flexible coupling provides a spacing, measured along a surface of the electrode array assembly, between the first and second electrode array subassemblies.
Aspect 2: The electrode array assembly of aspect 1, wherein the at least one electrode of each of the first and second electrode array subassemblies comprises a plurality of electrodes.
Aspect 3: The electrode array assembly of aspect 1 or aspect 2, wherein the flexible coupling provides a fixed, non-adjustable spacing, measured along the surface of the electrode array assembly, between the first and second electrode array subassemblies.
Aspect 4: The electrode array assembly of aspect 3, wherein the fixed, non-adjustable spacing is a patient-specific spacing.
Aspect 5: The electrode array assembly of aspect 3, wherein the fixed, non-adjustable spacing is a patient-specific spacing that positions the first and second electrode array subassemblies over a first and second target location in or on the patient or over opposite sides of the same target location in or on the patient.
Aspect 6: The electrode array assembly of aspect 1 or aspect 2, wherein the flexible coupling permits adjustment of the spacing, measured along the surface of the electrode array assembly, between the first and second electrode array subassemblies.
Aspect 7: The electrode array assembly of aspect 6, wherein the flexible coupling comprises an adjustable strap.
Aspect 8: The electrode array assembly of aspect 7, wherein the flexible coupling comprises at least one of:
an adjustable slide strap adjuster; or
hook material on a first portion of the flexible coupling and loop material on a second portion of the flexible coupling.
Aspect 9: The electrode array assembly of any one of the preceding aspects, wherein the flexible coupling has a length that permits simultaneous positioning of the first electrode array subassembly on a front portion of a torso of a patient and the second electrode array subassembly on a back portion of the torso of the patient.
Aspect 10: The electrode array assembly of any one of the preceding aspects, wherein the spacing, measured along the surface of the electrode array assembly, between the first and second electrode array subassemblies is from about 0.05 m to about 1.5 m.
Aspect 11: The electrode array assembly of any one of the preceding aspects, further comprising a first electrical lead and a second electrical lead, wherein each electrode of the at least one electrode of the first electrode array subassembly is in electrical communication with the first electrical lead, and wherein each electrode of the at least one electrode of the second electrode array subassembly is in electrical communication with the second electrical lead.
Aspect 12: The electrode array assembly of any one of the preceding aspects, wherein each of the first and second electrode array subassemblies comprises a flexible circuit that extends between each electrode of the at least one electrode of the electrode array subassembly.
Aspect 13: The electrode array assembly of any one of the preceding aspects, wherein the flexible coupling comprises a cover material that extends over an outwardly facing side of each of the first and second electrode array subassemblies.
Aspect 14: The electrode array assembly of any one of the preceding aspects, wherein the spacing, measured along the surface of the electrode array assembly, between the first and second electrode array subassemblies is a shortest distance, measured along the surface of the electrode array assembly, between any electrode of the at least one electrode of the first electrode array subassembly and any electrode of the at least one electrode of the second electrode array subassembly.
Aspect 15: The electrode array assembly of any one of the preceding aspects, wherein each of the first and second electrode array subassemblies comprises a skin contact layer that is configured to be positioned between the at least one electrode of the respective array subassembly and skin of a patient.
Aspect 16: The electrode array assembly of aspect 15, wherein the skin contact layer of each of the first and second electrode array subassemblies comprises conductive gel or conductive adhesive.
Aspect 17: The electrode array assembly of aspect 15, wherein the skin contact layer of each of the first and second electrode array subassemblies comprises a conductive adhesive.
Aspect 18: The electrode array assembly of any one aspects 15-17, wherein each of the first and second electrode array subassemblies comprises a dielectric material positioned between the electrode and the skin contact layer.
Aspect 19: The electrode array assembly of aspect 18, wherein the dielectric material comprises a polymer.
Aspect 20: The electrode array assembly of aspect 18, wherein the dielectric material comprises a ceramic.
Aspect 21: The electrode array assembly of any one of aspects 1-17, wherein at least one of the first and second electrode array subassemblies does not comprise a dielectric material between the electrode and the skin contact layer.
Aspect 22: The electrode array assembly of any one of the preceding aspects, wherein each of the first and second electrode array subassemblies comprises:
a layer of anisotropic material having a skin-facing surface and an opposing outwardly facing surface; and
a skin contact layer,
wherein the at least one electrode is in electrical contact with the outwardly facing surface of the layer of anisotropic material, and
wherein the skin contact layer is disposed on a skin-facing side of the layer of anisotropic material.
Aspect 23: The electrode array assembly of aspect 22, wherein the layer of anisotropic material is a synthetic graphite.
Aspect 24: The electrode array assembly of any one of aspects 22-23, wherein the layer of anisotropic material is a sheet of pyrolytic graphite.
Aspect 25: The electrode array assembly of any one of aspects 22-24, wherein the layer of anisotropic material is graphitized polymer film or graphite foil made from compressed high purity exfoliated mineral graphite.
Aspect 26: The electrode array assembly of any one of aspects 22-25, wherein the layer of anisotropic material is a first layer of anisotropic material, wherein each of the first and second electrode array subassemblies comprises:
a second layer of anisotropic material; and
a dielectric between the first and second layers of anisotropic material.
Aspect 27: The electrode array assembly of any one of aspects 22-26, wherein the layer of anisotropic material has a first thermal conductivity in a direction that is perpendicular to a plane of the layer, and wherein thermal conductivity of the layer in directions that are parallel to the plane of the layer is more than two times higher, or more than 10 times higher than the first thermal conductivity.
Aspect 28: The electrode array assembly of any one of aspects 22-27, wherein the layer of anisotropic material has a first resistance in a direction that is perpendicular to a plane of the layer, and wherein resistance of the layer in directions that are parallel to the plane of the layer is less than half, or less than 10% of the first resistance.
Aspect 29: The electrode array assembly of any one of aspects 22-28, wherein the skin contact layer is disposed on the skin-facing surface of the layer of anisotropic material.
Aspect 30: The electrode array assembly of any one of the preceding aspects, further comprising an upper adhesive layer comprising a conductive adhesive composite wherein the upper adhesive layer is disposed on an outwardly facing side of the layer of anisotropic material.
Aspect 31: The electrode array assembly of any one of the preceding aspects, further comprising at least one additional strap extending between the first and second electrode array subassemblies.
Aspect 32: The electrode array subassembly of aspect 31, wherein the at least one additional strap is coupled to the first and second electrode array subassemblies.
Aspect 33: The electrode array subassembly of aspect 31, wherein the at least one additional strap compressively retains the first and second electrode array subassemblies against the patient.
Aspect 34: A system comprising:
a plurality of electrode array assemblies including at least a first electrode assembly and a second electrode assembly, each of the plurality of electrode array assemblies comprising at least a first electrode array subassembly and a second electrode array subassembly and a flexible coupling extending between, and coupled to, the first and second electrode array subassemblies; and
at least one additional strap extending between the first electrode array subassembly of the first electrode assembly and the first electrode array subassembly of the second electrode assembly;
wherein each of the first and second electrode array subassemblies comprises at least one electrode; and wherein the flexible coupling provides a spacing, measured along a surface of the electrode array assembly, between the first and second electrode array subassemblies.
Aspect 35: The system of aspect 34, wherein the at least one additional strap extends compressively around a torso of a patient and retains at least the first electrode array subassembly of the first electrode assembly and the first electrode array subassembly of the second electrode assembly against the torso of the patient.
Aspect 36: A method comprising:
positioning the electrode array assembly of any one of the preceding aspects on a body of a patient.
Aspect 37: The method of aspect 36, wherein positioning the electrode array assembly comprises:
applying the first electrode array subassembly on a first location of the body of the patient; and
applying the second electrode array subassembly on a second location of the body of the patient that is spaced along the surface of the body by the spacing, measured along the surface of the electrode array assembly, between the first and second electrode subassemblies.
Aspect 38: The method of aspect 37, wherein, upon applying the second electrode assembly, the flexible coupling contacts the body of the patient along substantially an entire length of the flexible coupling.
Aspect 39: The method of aspect 37 or aspect 38, wherein the first location is on a front of a torso of the body of the patient, and wherein the second location is on a back of the torso of the body of the patient.
Aspect 40: The method of any one of aspects 36-39, wherein the flexible coupling comprises an adjustable strap, the method further comprising adjusting a length of the adjustable strap.
Aspect 41: The method of any one of aspects 36-40, further comprising applying an electric field between the at least one electrode of the first electrode array subassembly and the at least one electrode of the second electrode array subassembly.
Aspect 42: The method of aspect 41, wherein applying the electric field comprises applying an alternating electric field having an alternating current waveform at frequencies in a range from about 50 kHz to about 1 MHz and generating an electric field having an electric field intensity in the range of about 0.1 V/cm to about 10 V/cm.
Aspect 43: An electrode array assembly comprising:
a plurality of electrode array subassemblies, the plurality of electrode array subassemblies comprising at least a first electrode array subassembly and a second electrode array subassembly, wherein each electrode array subassembly of the plurality of electrode array subassemblies comprises at least one electrode; and
a flexible coupling extending between, and coupled to, the first and second electrode array subassemblies, wherein the flexible coupling provides a spacing, measured along a surface of the electrode array assembly, between the first and second electrode array subassemblies.
Aspect 44: A method comprising:
positioning, on a body of a patient, an electrode array assembly comprising:
a plurality of electrode array subassemblies, the plurality of electrode array subassemblies comprising at least a first electrode array subassembly and a second electrode array subassembly, wherein each electrode array subassembly of the plurality of electrode array subassemblies comprises at least one electrode; and
a flexible coupling extending between, and coupled to, the first and second electrode array subassemblies, wherein the flexible coupling provides a spacing, measured along a surface of the electrode array assembly, between the first and second electrode array subassemblies.
Aspect 45: The method of aspect 44, wherein positioning the electrode array assembly comprises:
applying the first electrode array subassembly on a first location of the body of the patient; and
applying the second electrode array subassembly on a second location of the body of the patient that is spaced along the surface of the body by the spacing, measured along the surface of the electrode array assembly, between the first and second electrode subassemblies.
Aspect 46: The method of aspect 45, wherein, upon applying the second electrode assembly, the flexible coupling contacts the body of the patient along substantially an entire length of the flexible coupling.
Aspect 47: The method of aspect 45 or aspect 46, wherein the first location is on a front of a torso of the body of the patient, and wherein the second location is on a back of the torso of the body of the patient.
Aspect 48: The method of any one of aspects 45-47, wherein the flexible coupling comprises an adjustable strap, the method further comprising adjusting a length of the adjustable strap.
Aspect 49: The method of any one of aspects 45-48, further comprising applying an electric field between the at least one electrode of the first electrode array subassembly and the at least one electrode of the second electrode array subassembly.
Aspect 50: The method of aspect 49, wherein applying the electric field comprises applying an alternating electric field having an alternating current waveform at frequencies in a range from about 50 kHz to about 1 MHz.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An electrode array assembly comprising:
first and second electrode array subassemblies, each of the first and second electrode array subassemblies comprising at least one electrode; and
a flexible coupling extending between, and coupled to, the first and second electrode array subassemblies, wherein the flexible coupling provides a spacing, measured along a surface of the electrode array assembly, between the first and second electrode array subassemblies.

2. The electrode array assembly of claim 1, wherein the at least one electrode of each of the first and second electrode array subassemblies comprises a plurality of electrodes.

3. The electrode array assembly of claim 1, wherein the flexible coupling provides a fixed, non-adjustable spacing, measured along the surface of the electrode array assembly, between the first and second electrode array subassemblies.

4. The electrode array assembly of claim 3, wherein the fixed, non-adjustable spacing is a patient-specific spacing.

5. The electrode array assembly of claim 3, wherein the fixed, non-adjustable spacing is a patient-specific spacing that positions the first and second electrode array subassemblies over a first and second target location in or on the patient or over opposite sides of the same target location in or on the patient.

6. The electrode array assembly of claim 1, wherein the flexible coupling permits adjustment of the spacing, measured along the surface of the electrode array assembly, between the first and second electrode array subassemblies.

7. The electrode array assembly of claim 6, wherein the flexible coupling comprises an adjustable strap.

8. The electrode array assembly of claim 7, wherein the flexible coupling comprises at least one of:
an adjustable slide strap adjuster; or
hook material on a first portion of the flexible coupling and loop material on a second portion of the flexible coupling.

9. The electrode array assembly of claim 1, wherein the flexible coupling has a length that permits simultaneous positioning of the first electrode array subassembly on a front portion of a torso of a patient and the second electrode array subassembly on a back portion of the torso of the patient.

10. The electrode array assembly of claim 1, wherein the spacing, measured along the surface of the electrode array assembly, between the first and second electrode array subassemblies is from about 0.05 m to about 1.5 m.

11. The electrode array assembly of claim 1, further comprising at least one additional strap extending between the first and second electrode array subassemblies.

12. The electrode array subassembly of claim 11, wherein the at least one additional strap is coupled to the first and second electrode array subassemblies.

13. The electrode array subassembly of claim 11, wherein the at least one additional strap compressively retains the first and second electrode array subassemblies against the patient.

14. A system comprising:
a plurality of electrode array assemblies including at least a first electrode assembly and a second electrode assembly, each of the plurality of electrode array assemblies comprising at least a first electrode array subassembly and a second electrode array subassembly and a flexible coupling extending between, and coupled to, the first and second electrode array subassemblies; and
at least one additional strap extending between the first electrode array subassembly of the first electrode assembly and the first electrode array subassembly of the second electrode assembly;
wherein each of the first and second electrode array subassemblies comprises at least one electrode; and wherein the flexible coupling provides a spacing, measured along a surface of the electrode array assembly, between the first and second electrode array subassemblies.

15. The system of claim 14 wherein the at least one additional strap extends compressively around a torso of a patient and retains at least the first electrode array subassembly of the first electrode assembly and the first electrode array subassembly of the second electrode assembly against the torso of the patient.

16. A method comprising:
positioning, on a body of a patient, an electrode array assembly comprising:
a plurality of electrode array subassemblies, the plurality of electrode array subassemblies comprising at least a first electrode array subassembly and a second electrode array subassembly, wherein each electrode array subassembly of the plurality of electrode array subassemblies comprises at least one electrode; and
a flexible coupling extending between, and coupled to, the first and second electrode array subassemblies, wherein the flexible coupling provides a spacing, measured along a surface of the electrode array assembly, between the first and second electrode array subassemblies.

17. The method of claim 16, wherein positioning the electrode array assembly comprises:
applying the first electrode array subassembly on a first location of the body of the patient; and
applying the second electrode array subassembly on a second location of the body of the patient that is spaced along the surface of the body by the spacing, measured along the surface of the electrode array assembly, between the first and second electrode subassemblies.

18. The method of claim 17, wherein, upon applying the second electrode assembly, the flexible coupling contacts the body of the patient along substantially an entire length of the flexible coupling.

19. The method of claim 17, wherein the first location is on a front of a torso of the body of the patient, and wherein the second location is on a back of the torso of the body of the patient.

20. The method of claim 16, wherein the flexible coupling comprises an adjustable strap, the method further comprising adjusting a length of the adjustable strap.

21. The method of claim 16, further comprising applying an electric field between the at least one electrode of the first electrode array subassembly and the at least one electrode of the second electrode array subassembly, wherein applying the electric field comprises applying an alternating electric field having an alternating current waveform at frequencies in a range from about 50 kHz to about 1 MHz.
